(19) United States
(12) Patent Application Publication
(10) Pub. No.: US 2007/0060655 A1
(43) Pub. Date: Mar. 15, 2007

(54) Use of agomelatine in obtaining medicaments intended for the treatment of sleep disorders in the depressed patient

(75) Inventor: Bruno Delalleau, Asnieres-Sur-Seine (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Courbevoie (FR)

(21) Appl. No.: 11/517,520

(22) Filed: Sep. 7, 2006

(30) Foreign Application Priority Data
Sep. 9, 2005 (FR) 05.09207

(51) Int. Cl. A61K 31/16

(52) U.S. Cl. 514/630

(57) ABSTRACT

The present invention relates to the use of agomelatine, or N-[2-(7-methoxy-1-naphthyl)ethyl]acetamide, in obtaining medicaments intended for the treatment of sleep disorders in the depressed patient.

The present invention relates to the use of agomelatine, or N-[2-(7-methoxy-1-naphthyl)ethyl]acetamide of formula (I): 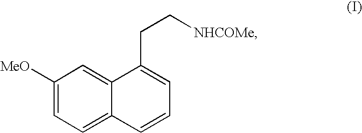
or its hydrates, crystalline forms and addition salts with a pharmaceutically acceptable acid or base, alone or in association, in obtaining medicaments intended for the treatment of sleep disorders in the depressed patient.

Agomelatine, or N-[2-(7-methoxy-1-naphthyl)ethyl]acetamide, has the double characteristic of being, on the one hand, an agonist of receptors of the melatoninergic system and, on the other hand, an antagonist of the 5-HT_{2c} receptor. These properties provide it with activity in the central nervous system and, more especially, in the treatment of major depressive disorder, seasonal affective disorder, sleep disorders, cardiovascular pathologies, pathologies of the digestive system, insomnia and fatigue due to jet-lag, and appetite disorders and obesity.

Agomelatine, its preparation and its use in therapeutics have been described in European Patent Specification EP 0 447 285.

The Applicant has now found that agomelatine, or N-[2-(7-methoxy-1-naphthyl)ethyl]-acetamide or its hydrates, crystalline forms and addition salts with a pharmaceutically acceptable acid or base, has valuable properties allowing its use in the treatment of sleep disorders in the depressed patient.

Sleep is the most prominent circadian biological rhythm in human beings. That rhythm is seriously disturbed in the depressed patient and there is currently no satisfactory and recognised treatment for alleviating those disorders. The current antidepressant treatments (fluoxetine, paroxetine, venlafaxine . . . ) have little or no specific effect on sleep disorders in the depressed patient. Those which are active are so as a result of a powerful and non-specific sedative action that leads to a decrease in the diurnal cognitive capacities of the subjects treated. This is true for mianserin or mirtazapine, for example (Ridout et al. 2001, Fawcett et al. 1998).

The Applicant has now discovered that agomelatine is especially well suited to the treatment of sleep disorders in the depressed patient. Indeed, improvement of sleep in the depressed patient need not be to the detriment of sleep structure: antidepressants with non-specific sedative action disturb that structure, especially by altering paradoxal sleep or slow deep sleep (Zarifian et al. 1982). In contrast to that, the Applicant has now discovered that agomelatine does not behave like a conventional antidepressant, its action respecting sleep structure and thus the homeostatic sleep response in the depressed patient. Those results allow its use, even its prolonged use, to be considered in sleep disorders in the depressed patient.

The invention accordingly relates to the use of agomelatine or its hydrates, crystalline forms and addition salts with a pharmaceutically acceptable acid or base, in obtaining pharmaceutical compositions intended for the treatment of sleep disorders in the depressed patient.

The pharmaceutical compositions will be presented in forms suitable for administration by the oral, parenteral, transcutaneous, nasal, rectal or perlingual route, and especially in the form of injectable preparations, tablets, sublingual tablets, glossettes, gelatin capsules, capsules, lozenges, suppositories, creams, ointments, dermal gels etc.

Besides agomelatine, the pharmaceutical compositions according to the invention comprise one or more excipients or carriers selected from diluents, lubricants, binders, disintegration agents, absorbents, colourants, sweeteners etc.

By way of example, and without implying any limitation, there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: aluminium and magnesium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the sex, age and weight of the patient, the administration route, the nature of the disorder and any associated treatments and ranges from 1 mg to 50 mg of agomelatine per 24 hours.

The daily dose of agomelatine will preferably be 25 mg per day.

Pharmaceutical Composition:

Formula for the preparation of 1000 tablets each containing 25 mg of active ingredient:

| | | |
|---|---|---|
| Agomelatine | 25 | g |
| Lactose monohydrate | 62 | g |
| Magnesium stearate | 1.3 | g |
| Povidone | 9 | g |
| Anhydrous colloidal silica | 0.3 | g |
| Cellulose sodium glycolate | 30 | g |
| Stearic acid | 2.6 | g |

Clinical Studies:

The action specific to agomelatine on sleep disorders in the depressed patient is established in comparison with mirtazapine. This study, carried out as a double-blind over 6 weeks of treatment, has, as its principal criterion of efficacy, the polysomnographic recording of sleep. The Hamilton Scale of Depression allows the antidepressant efficacy to be documented. The results obtained show that agomelatine is especially effective in the treatment of sleep disorders in the depressed patient.

Another study evaluating sleep improvement was carried out in patients with a characterised depressive state as a double blind over 6 weeks compared with venlafaxine. After one week placebo, 332 patients were randomised: 165 received 25 mg/day of agomelatine and 167 received 75 mg/day of venlafaxine for 2 weeks. After 2 weeks of treatment, if clinical improvements obtained were found insufficient, the doses for some patients were doubled to 50 mg/day of agomelatine or 150 mg/day of venlafaxine for the following weeks.

The Hamilton Scale of Depression allows the antidepressant efficacy to be documented, and the results obtained demonstrated a comparable efficacy of agomelatine versus venlafaxine.

The effects on sleep were evaluated using the Leeds Sleep Evaluation Questionnaire (LSEQ): in the item “ease of getting to sleep” (GTS), effects of agomelatine have been more important and faster to appear than for venlafaxine with a significant difference from the first week (p=0.007) which continue in favour of agomelatine during the 6 weeks of treatment. Regarding the item “quality of sleep” (QOS), agomelatine demonstrated appreciably greater efficacy (p=0.015) than venlafaxine. An equally significant improvement (p less than or equal to 0.001) in favour of agomelatine versus venlafaxine was reported for the phenomena of “sleepiness during the day” and “sensation of well-being”.

Those results attest to the superiority of the efficacy of agomelatine in the treatment of sleep disorders in the depressed patient.

Lastly, effects of agomelatine on the sleep polysomnographic parameters were evaluated in a pilot study carried out in 15 patients with a characterised depressive state. Restoration of the sleep physiological structure was observed with agomelatine 25 mg. The absolute and relative durations of cycles 3 and 4 of the recording of the electroencephalographic activity are significantly improved without alteration of the REM (rapid eye movements) sleep. Furthermore, agomelatine increase the total duration of sleep, reduce the number of nocturnal wakenings, and as a consequence improve sleep efficacy. Those beneficial effects were observed from the first week of treatment.

1- A method for treating a living animal body, including a human, afflicted with sleep disorders in the depressed patient comprising the step of administering to the living animal body, including a human, an amount of agomelatine, or N-[2-(7-methoxy-1-naphthyl)ethyl]acetamide or hydrates, crystalline forms and addition salts thereof with a pharmaceutically acceptable acid or base which is effective for alleviation of sleep disorders in the depressed patient.

2- A pharmaceutical composition comprising agomelatine, or N-[2-(7-methoxy-l-naphthyl)ethyl]acetamide or hydrates, crystalline forms and addition salts thereof with a pharmaceutically acceptable acid or base, alone or in combination with one or more pharmaceutically acceptable excipients.
